(19) United States
(12) Patent Application Publication
(10) Pub. No.: US 2003/0013951 A1
(43) Pub. Date: Jan. 16, 2003

(54) Database organization and searching

(76) Inventors: Dan Stefanescu, Needham, MA (US); Michael Leventon, Lexington, MA (US)

Correspondence Address: ROPES & GRAY, ONE INTERNATIONAL PLACE, BOSTON, MA, 02110-2624, US

(21) Appl. No.: 09/960,874

(22) Filed: Sep. 21, 2001

Related U.S. Application Data
(60) Provisional application No. 60/234,108, filed on Sep. 21, 2000.
(60) Provisional application No. 60/234,435, filed on Sep. 21, 2000.
(60) Provisional application No. 60/234,114, filed on Sep. 21, 2000.
(60) Provisional application No. 60/234,115, filed on Sep. 21, 2000.

(51) Int. Cl. A61B 5/05

(52) U.S. Cl. 600/407

(57) ABSTRACT

The database organization and searching systems disclosed herein provide techniques for organizing large-scale image data sources such as medical image databases. Database records such as medical images may be pre-processed, such as through registration, segmentation, and extraction of feature vectors, to effectively normalize data among different images. Each image, or a portion thereof, is then labeled according to some observed characteristic or other attribute. A model, such as a linear regression model, may then be trained to associate the feature vectors with the labels. The model is then available for labeling other images. In this manner, search techniques for well-organized or indexed databases may be applied automatically to databases that are not well-organized, but that have the same underlying data type. Data that is organized in this way may also be used to construct diagnostic aids or other tools.

### CROSS-REFERENCE TO RELATED APPLICATIONS

This application claims the benefit of, and incorporates by reference, the entire disclosure of U.S. Provisional Patent Application No. 60/234,108 filed on Sep. 21, 2000, U.S. Provisional Patent Application No. 60/234,435, filed on Sep. 21, 2000, U.S. Provisional Patent Application No. 60/234,114, filed on Sep. 21, 2000, and U.S. Provisional Patent Application No. 60/234,115, filed on Sep. 21, 2000.

### FIELD OF THE INVENTION

The invention relates generally to databases, and more particularly to organizing and searching database of medical images.

### BACKGROUND OF THE INVENTION

Significant progress has been made in the capture of medical images, such as CT scans, x-ray images, ultrasound images, magnetic resonance imaging (MRI) and so forth. At the same time, computer-assisted evaluation of images has moved forward with a number of different computational techniques, further aided by continuing improvements in computer processing power and data networking.

Nonetheless, practical applications for computer-assisted evaluation of medical images have proved elusive. For example, in neural radiology, a single three-dimensional, neural MR study may contain large amounts of data, and a database of such studies may consume terabytes of storage or more. Furthermore, from an image-processing point of view, this image-based data is characterized by significant complexity that makes accurate matching a difficult and computationally expensive task, for traditional, state-of-the-art technology. Thus, while processing and matching strategies may be devised for such images using known techniques, these techniques have thus far failed to produce systems suitable for deployment on typical desktop computers and network connections.

There remains a need for systems that provide management and evaluation tools for large-scale image databases, such as medical image databases.

### SUMMARY OF THE INVENTION

The database organization and searching systems disclosed herein provide techniques for organizing large-scale image data sources such as medical image databases. Database records such as medical images may be pre-processed, such as through registration, segmentation, and extraction of feature vectors, to effectively normalize data among different images. Each image, or a portion thereof, is then labeled according to some observed characteristic or other attribute. A model, such as a linear regression model, may then be trained to associate the feature vectors with the labels. The model is then available for labeling other images. In this manner, search techniques for well-organized or indexed databases may be applied automatically to databases that are not well-organized, but that have the same underlying data type. Data that is organized in this way may also be used to construct diagnostic aids or other tools.

### BRIEF DESCRIPTION OF DRAWINGS

The foregoing and other objects and advantages of the invention will be appreciated more fully from the following further description thereof, with reference to the accompanying drawings, wherein:

FIG. 1 shows a schematic diagram of the entities involved in an embodiment of a method and system disclosed herein;

FIG. 2 shows a block diagram of a server that may be used with the systems described herein;

FIG. 3 shows a page that may be used as a user interface;

FIG. 4 shows a patient workspace of a user interface;

FIG. 5 shows an atlas workspace of a user interface;

FIG. 6 shows a reference workspace of a user interface;

FIG. 7 shows a results workspace of a user interface;

FIG. 8 is a flow chart showing a process for processing images according to the systems described herein;

FIG. 9 shows several possible arrangements of tiles for sampling a magnetic resonance image;

FIG. 10 is a flowchart of a process for organizing databases;

FIG. 11 shows a state diagram for a workflow management system;

FIG. 12 shows schematically a parameter space of a database and of users having overlapping interests;

FIG. 13 shows a process flow for entering user data to form/join a group;

FIG. 14 depicts exemplary data entries in user data sheet;

FIG. 15 depicts handling of user data without a match in the database; and

FIG. 16 shows an exemplary process for matching patient data with clinical trials.

### DETAILED DESCRIPTION OF CERTAIN EMBODIMENTS

To provide an overall understanding of the invention, certain illustrative embodiments will now be described, including a client/server architecture for a medical image processing system. However, it will be understood that the methods and systems described herein can be suitably adapted to any environment where image data or other complex data structures are to be organized into a database for modeling, searching, or other further processing, and may be deployed, for example, as a stand-alone desktop computer application, within a corporate intranet or over a private network. Applications of the organized database may include, for example, medical diagnosis, statistical analysis, or preparation of instructional, academic software. These and other applications of the systems described herein are intended to fall within the scope of the invention. More generally, the principles of the invention are generally applicable to any environment where organization or analysis of image data or other complex data structures is desired.

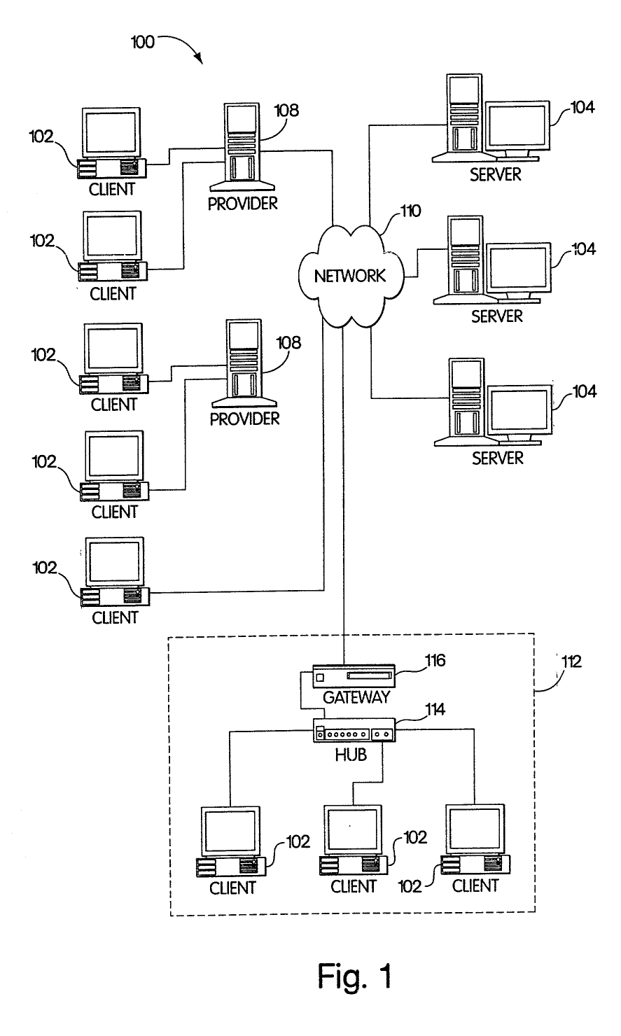

FIG. 1 shows a schematic diagram of the entities involved in an embodiment of a method and system disclosed herein. In a system 100, a plurality of clients 102, servers 104, and providers 108 are connected via an internetwork 110. It should be understood that any number of clients 102, servers 104, and providers 108 could participate in such a system 100. The system may further include one or more local area networks (“LAN”) 112 interconnecting clients 102 through a hub 114 (in, for example, a peer network such as Ethernet) or a local area network server 114 (in, for example, a client-server network). The LAN 112 may be connected to the internetwork 110 through a gateway 116, which provides security to the LAN 112 and ensures operating compatibility between the LAN 112 and the internetwork 110. Any data network may be used as the internetwork 110 and the LAN 112.

In one embodiment, the internetwork 110 is the Internet, and the World Wide Web provides a system for interconnecting clients 102 and servers 104 through the Internet 110.

The internetwork 110 may include a cable network, a wireless network, and any other networks for interconnecting clients, servers and other devices.

An exemplary client 102 includes the conventional components of a client system, such as a processor, a memory (e.g. RAM), a bus which couples the processor and the memory, a mass storage device (e.g. a magnetic hard disk or an optical storage disk) coupled to the processor and the memory through an I/O controller, and a network interface coupled to the processor and the memory, such as modem, digital subscriber line (“DSL”) card, cable modem, network interface card, wireless network card, or other interface device capable of wired, fiber optic, or wireless data communications. One example of such a client 102 is a personal computer equipped with an operating system such as Microsoft Windows 2000, Microsoft Windows NT, Unix, Linux, and Linux variants, along with software support for Internet communication protocols. The personal computer may also include a browser program, such as Microsoft Internet Explorer or Netscape Navigator, to provide a user interface for access to the Internet 110. Although the personal computer is a typical client 102, the client 102 may also be a workstation, mobile computer, Web phone, television set-top box, interactive kiosk, personal digital assistant, or other device capable of communicating over the Internet 110. As used herein, the term “client” is intended to refer to any of the above-described clients 102, as well as proprietary network clients designed specifically for the medical image processing systems described herein, and the term “browser” is intended to refer to any of the above browser programs or other software or firmware providing a user interface for navigating the Internet 110 and/or communicating with the medical image processing systems.

An exemplary server 104 includes a processor, a memory (e.g. RAM), a bus which couples the processor and the memory, a mass storage device (e.g. a magnetic or optical disk) coupled to the processor and the memory through an I/O controller, and a network interface coupled to the processor and the memory. Servers may be organized as layers of clusters in order to handle more client traffic, and may include separate servers for different functions such as a database server, a file server, an application server, and a Web presentation server. Such servers may further include one or more mass storage devices such as a disk farm or a redundant array of independent disk (“RAID”) system for additional storage and data integrity. Read-only devices, such as compact disc drives and digital versatile disc drives, may also be connected to the servers. Suitable servers and mass storage devices are manufactured by, for example, Compaq, IBM, and Sun Microsystems. As used herein, the term “server” is intended to refer to any of the above-described servers 104.

Focusing now on the internetwork 110, one embodiment is the Internet. The structure of the Internet 110 is well known to those of ordinary skill in the art and includes a network backbone with networks branching from the backbone. These branches, in turn, have networks branching from them, and so on. The backbone and branches are connected by routers, bridges, switches, and other switching elements that operate to direct data through the internetwork 110. For a more detailed description of the structure and operation of the Internet 110, one may refer to “The Internet Complete Reference,” by Harley Hahn and Rick Stout, published by McGraw-Hill, 1994. However, one may practice the present invention on a wide variety of communication networks. For example, the internetwork 110 can include interactive television networks, telephone networks, wireless data transmission systems, two-way cable systems, customized computer networks, interactive kiosk networks, or ad hoc packet relay networks.

One embodiment of the internetwork 110 includes Internet service providers 108 offering dial-in service, such as Microsoft Network, America OnLine, Prodigy and CompuServe. It will be appreciated that the Internet service providers 108 may also include any computer system which can provide Internet access to a client 102. Of course, the Internet service providers 108 are optional, and in some cases, the clients 102 may have direct access to the Internet 110 through a dedicated DSL service, ISDN leased lines, Ti lines, digital satellite service, cable modem service, or any other high-speed connection to a network point-of-presence. Any of these high-speed services may also be offered through one of the Internet service providers 108.

In its present deployment as the Internet, the internetwork 110 consists of a worldwide computer network that communicates using protocols such as the well-defined Transmission Control Protocol (“TCP”) and Internet Protocol (“IP”) to provide transport and network services. Computer systems that are directly connected to the Internet 110 each have a unique IP address. The IP address consists of four one-byte numbers (although a planned expansion to sixteen bytes is underway with IPv6). The four bytes of the IP address are commonly written out separated by periods such as “12.30.58.7”. To simplify Internet addressing, the Domain Name System (“DNS”) was created. The DNS allows users to access Internet resources with a simpler alphanumeric naming system. A DNS name consists of a series of alphanumeric names separated by periods. For example, the name “www.mdol.com” corresponds to a particular IP address. When a domain name is used, the computer accesses a DNS server to obtain the explicit four-byte IP address. It will be appreciated that other internetworks 110 may be used with the invention. For example, the internetwork 110 may be a wide-area network, a local area network, or corporate area network.

To further define the resources on the Internet 110, the Uniform Resource Locator system was created. A Uniform Resource Locator (“URL”) is a descriptor that specifically defines a type of Internet resource along with its location. URLs have the following format:

resource-type://domain.address/path-name

where resource-type defines the type of Internet resource. Web documents are identified by the resource type “http” which indicates that the hypertext transfer protocol should be used to access the document. Other common resource types include “ftp” (file transmission protocol), “mailto” (send electronic mail), “file” (local file), and “telnet.” The domain.address defines the domain name address of the computer that the resource is located on. Finally, the path-name defines a directory path within the file system of the server that identifies the resource. As used herein, the term “IP address” is intended to refer to the four-byte Internet Protocol address (or the sixteen-byte IPv6 address), and the term “Web address” is intended to refer to a domain name address, along with any resource identifier and path name appropriate to identify a particular Web resource. The term “address,” when used alone, is intended to refer to either a Web address or an IP address.

In an exemplary embodiment, a browser, executing on one of the clients 102, retrieves a Web document at an address from one of the servers 104 via the internetwork 110, and displays the Web document on a viewing device, e.g., a screen. A user can retrieve and view the Web document by entering, or selecting a link to, a URL in the browser. The browser then sends an http request to the server 104 that has the Web document associated with the URL. The server 104 responds to the http request by sending the requested Web document to the client 102. The Web document is an HTTP object that includes plain text (ASCII) conforming to the HyperText Markup Language (“HTML”). Other markup languages are known and may be used on appropriately enabled browsers and servers, including the Dynamic HyperText Markup Language (“DHTML”), the Extensible Markup Language (“XML”), the Extensible Hypertext Markup Language (“XHML”), and the Standard Generalized Markup Language (“SGML”).

Each Web document may contains hyperlinks to other Web documents. The browser displays the Web document on the screen for the user and the hyperlinks to other Web documents are emphasized in some fashion such that the user can identify and select each hyperlink. To enhance functionality, a server 104 may execute programs associated with Web documents using programming or scripting languages, such as Perl, C, C++, or Java. A server 104 may also use server-side scripting languages such as ColdFusion from Allaire, Inc., or PHP. These programs and languages perform “back-end” functions such as order processing, database management, and content searching. A Web document may also include references to small client-side applications, or applets, that are transferred from the server 104 to the client 102 along with a Web document and executed locally by the client 102. Java is one popular example of a programming language used for applets. The text within a Web document may further include (non-displayed) scripts that are executable by an appropriately enabled browser, using a scripting language such as JavaScript or Visual Basic Script. Browsers may further be enhanced with a variety of helper applications to interpret various media including still image formats such as JPEG and GIF, document formats such as PS and PDF, motion picture formats such as AVI and MPEG, and sound formats such as MP3 and MIDI. These media formats, along with a growing variety of proprietary media formats, may be used to enrich a user's interactive and audio-visual experience as each Web document is presented through the browser. The term “page” as used herein is intended to refer to the Web document described above, as well as any of the above-described functional or multimedia content associated with the Web document.

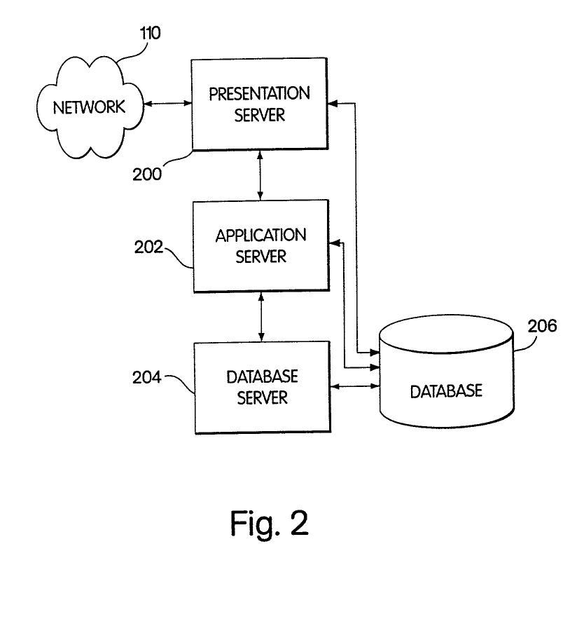

FIG. 2 shows a block diagram of a server that may be used with the systems described herein. In this embodiment, the server 104 includes a presentation server 200, an application server 202, and a database server 204. The application server 202 is connected to the presentation server 200. The database server 204 is also connected to the presentation server 200 and the application server 202, and is further connected to a database 206 embodied on a mass storage device. The presentation server 200 includes a connection to the internetwork 110. It will be appreciated that each of the servers may comprise more than one physical server, as required for capacity and redundancy, and it will be further appreciated that in some embodiments more than one of the above servers may be logical servers residing on the same physical device. It will further be appreciated that one or more of the servers may be at a remote location, and may communicate with the presentation server 200 through a local area or wide area network. The term “host,” as used herein, is intended to refer to any combination of servers described above that include a presentation server 200 for providing access to pages by the clients 102. The term “site,” as used herein, is intended to refer to a collection of pages sharing a common domain name address, or dynamically generated by a common host, or accessible through a common host (i.e., a particular page may be maintained on or generated by a remote server, but nonetheless be within a site).

The presentation server 200 provides an interface for one or more connections to the internetwork 110, thus permitting more than one of the clients 102 (FIG. 1) to access the site at the same time. In one embodiment, the presentation server 200 comprises a plurality of enterprise servers, such as the ProLiant Cluster available from Compaq Computer Corp., or a cluster of E250's from Sun MicroSystems running Solaris 2.7. Other suitable servers are known in the art and are described in Jamsa, Internet Programming, Jamsa Press (1995), the teachings of which are herein incorporated by reference. The server maintains one or more connections to the Internet 110, preferably provided by a tier one provider, i.e., one of the dozen or so national/international Internet backbones with cross-national links of T3 speeds or higher, such as MCI, UUNet, BBN Planet, and Digex. Each server may be, for example, an iPlanet Enterprise Server 4.0 from the Sun/Netscape Alliance. The presentation server 200 may also, for example, Microsoft's .NET technology, or use a Microsoft Windows operating system, with a “front end” written in Microsoft Active Server Page (“ASP”), or some other programming language or server software capable of integrating ActiveX controls, forms, Visual Basic Scripts, JavaScript, Macromedia Flash Technology multimedia, e-mail, and other functional and multimedia aspects of a page. Typically, the front end includes all text, graphics, and interactive objects within a page, along with templates used for dynamic page creation.

A client 102 (FIG. 1) accessing an address hosted by the presentation server 200 will receive a page from the presentation server 200 containing text, forms, scripts, active objects, hyperlinks, etc., which may be collectively viewed using a browser. Each page may consist of static content, i.e., an HTML text file and associated objects (*.avi, * jpg, *.gif, etc.) stored on the presentation server, and may include active content including applets, scripts, and objects such as check boxes, drop-down lists, and the like. A page may be dynamically created in response to a particular client 102 request, including appropriate queries to the database server 204 for particular types of data to be included in a responsive page. It will be appreciated that accessing a page is more complex in practice, and includes, for example, a DNS request from the client 102 to a DNS server, receipt of an IP address by the client 102, formation of a TCP connection with a port at the indicated IP address, transmission of a GET command to the presentation server 200, dynamic page generation (if required), transmission of an HTML object, fetching additional objects referenced by the HTML object, and so forth.

The application server 202 provides the “back-end” functionality of the Web site, and includes connections to the presentation server 200 and the database server 204. In one embodiment, the presentation server 200 comprises an enterprise server, such as one available from Compaq Computer Corp., running the Microsoft Windows NT operating system, or a cluster of E250's from Sun MicroSystems running Solaris 2.7. The back-end software may be implemented using pre-configured e-commerce software, such as that available from Pandesic, to provide back-end functionality including order processing, billing, inventory management, financial transactions, shipping instructions, and the like.

The e-commerce software running on the application server 202 may include a software interface to the database server 204, as well as a software interface to the front end provided by the presentation server 200. The application server 200 may also use a Sun/Netscape Alliance Server 4.0. A payment transaction server may also be included to process payments at a Web site using third party services such as Datacash or WorldPay, or may process payments directly using payment server and banking software, along with a communication link to a bank. While the above describes one form of application server that may be used with the systems described herein, other configurations are possible, as will be described in further detail below.

The database server 204 may be an enterprise server, such as one available from Compaq Computer Corp., running the Microsoft Windows NT operating system or a cluster of E250's from Sun MicroSystems running Solaris 2.7, along with software components for database management. Suitable databases are provided by, for example, Oracle, Sybase, and Informix. The database server 204 may also include one or more databases 206, typically embodied in a mass-storage device. The databases 206 may include, for example, user interfaces, search results, search query structures, lexicons, user information, and the templates used by the presentation server to dynamically generate pages. It will be appreciated that the databases 206 may also include structured or unstructured data, as well as storage space, for use by the presentation server 200 and the application server 202. In operation, the database management software running on the database server 204 receives properly formatted requests from the presentation server 200, or the application server 202. In response, the database management software reads data from, or writes data to, the databases 206, and generates responsive messages to the requesting server. The database server 204 may also include a File Transfer Protocol (“FTP”) or a Secure Shell (“SSH”) server for providing downloadable files.

While the three tier architecture described above is one conventional architecture that may be used with the systems described herein, it will be appreciated that other architectures for providing data and processing through a network are known and may be used in addition to, or in conjunction with, or in place of the described architecture. Any such system may be used, provided that it can support aspects of the image processing system described herein.

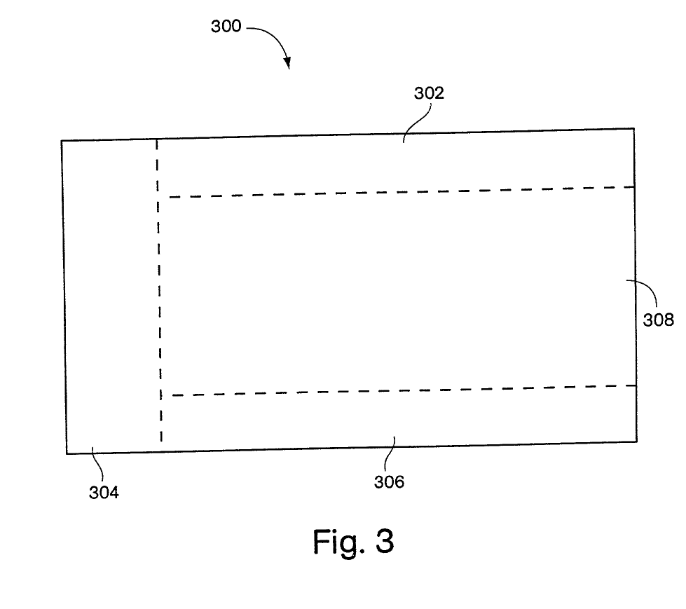

FIG. 3 shows a page that may be used as a user interface. The page 300 may include a header 302, a sidebar 304, a footer 306 and a main section 308, all of which may be displayed at a client 102 using a browser. The header 302 may include, for example, one or more banner advertisements and a title of the page. The sidebar 304 may include a menu of choices for a user at the client 102. The footer 306 may include another banner advertisement, and/or information concerning the site such as a “help” or “webmaster” contact, copyright information, disclaimers, a privacy statement, etc. The main section 308 may include content for viewing by the user. The main section 308 may also include, for example, tools for electronically mailing the page to an electronic mail (“e-mail”) account, searching content at the site, and so forth. It will be appreciated that the description above is generic, and may be varied according to where a client 102 is within a Web site related to the page, as well as according to any available information about the client 102 (such as display size, media capabilities, etc.) or the user.

A Web site including the page 300 may use cookies to track users and user information. In particular, a client 102 accessing the site may be accessed to detect whether the client 102 has previously accessed the page or the site. If the client 102 has accessed the site, then some predetermined content may be presented to the client 102. If the client 102 does not include a cookie indicating that the client 102 has visited the site, then the client 102 may be directed to a registration page where information may be gathered to create a user profile. The client 102 may also be presented with a login page, so that a pre-existing user on a new client 102 may nonetheless bypass the registration page.

The site may provide options to the client 102. For example, the site may provide a search tool by which the client 102 may search for content within the site, or content external to the site but accessible through the internetwork 110. The site may include news items topical to the site. Banner ads may be provided in the page 300, and the ads may be personalized to a client 102 if a profile exists for that client 102. The banner ads may also track redirection. That is, when a client 102 selects a banner ad, the link and the banner ad may be captured and stored in a database. The site may provide a user profile update tool by which the client 102 may make alterations to a user profile.

It will be appreciated that the foregoing description has been generic. A user interface for a medical image processing system will now be described in more detail. It will be appreciated that the interface may be embodied in any software and/or hardware client operating on a client device, including a browser along with any suitable plug-ins, a Java applet, a Java application, a C or C++ application, or any other application or group of applications operating on a client device. In one embodiment, the user interface may deployed through a Web browser. In one embodiment, the user interface may be deployed as an application running on a client device, with suitable software and/or hardware for access to an internetwork. In these and other embodiments, certain image processing functions may be distributed in any suitable manner between a client device and one or more servers, as will be explained in further detail below.

In the example embodiment described below, each workspace, including a patient workspace, an atlas workspace, a reference workspace, and a results workspace, may be selected using tabs such as those provided for Windows applications. Each workspace will now be explained in further detail.

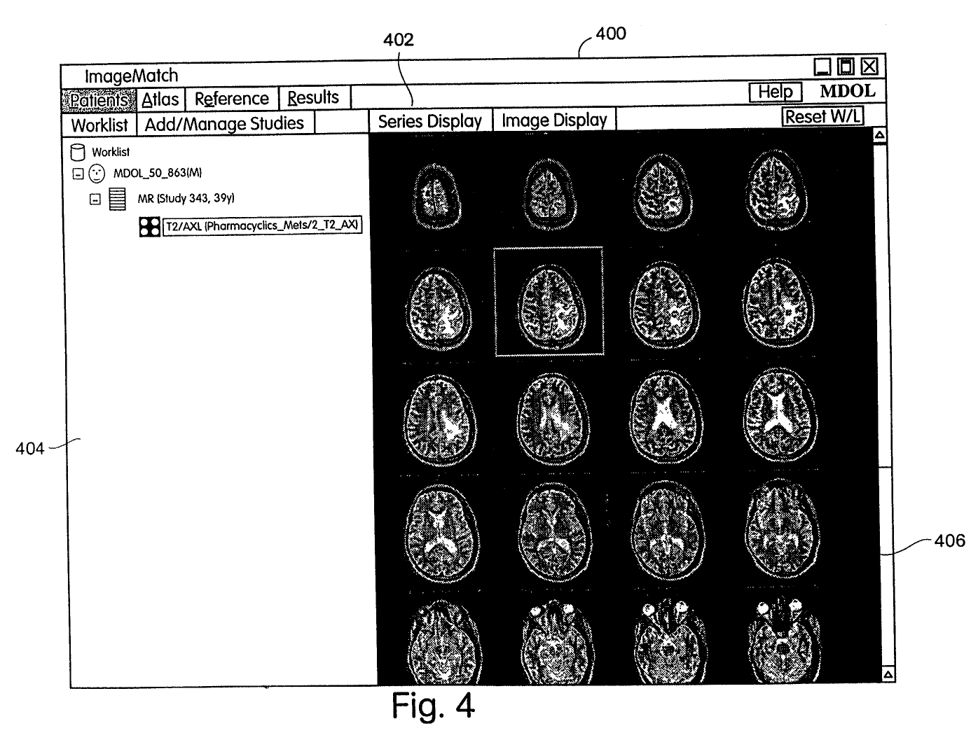

FIG. 4 shows a patient workspace of a user interface. As described above, the interface 400 may be deployed as an application running on a local machine, as a remote service run from an application service provider, as a Web-based resource accessible over an internetwork, or any other mode suitable for use at a client device. Functionality may be distributed in any suitable manner between the client device and one or more remote resources such as databases, servers, and the like. It will be appreciated that the interface 400 depicted in FIG. 4 is an example, and that other arrangements of the interface may be used consistent with the systems described herein. It will also be appreciated that menus, submenus, and other interface screens may be usefully employed to support the functionality of the interface, such as menus for controlling scoring and relevancy of search results.

A patient workspace 402 may include a worklist area 404 and an image display area 406, along with any suitable navigation aids, such as the vertical scroll bar depicted on the right hand side of the image display area 406. The worklist area 404 may include a button for accessing a worklist managing interface through which a user may add, remove, or otherwise manage and dispose of cases, including providing diagnostic conclusions and any other findings or observations. A worklist displayed in the worklist area may include one or more cases for review, such as neural radiology MR studies. Once a case has been reviewed and, for example, a diagnosis reached, the case may be dispatched from the worklist using the button.

The image display area 406 may display one or more images, such as a series of images in an MR study. Through the interface 400, a user may select on of the series of images for enlarged display. In this manner, a user may navigate through images such as provided in an MR study, and review in detail areas of possible interest. In one embodiment, a user may select one or more regions of interest graphically within the image display area 406. Regions of interest, such as possible pathologies or other abnormalities, may be demarcated as points, predetermined geometric shapes (e.g., squares, triangles, ellipses, etc.), or as hand-drawn contours.

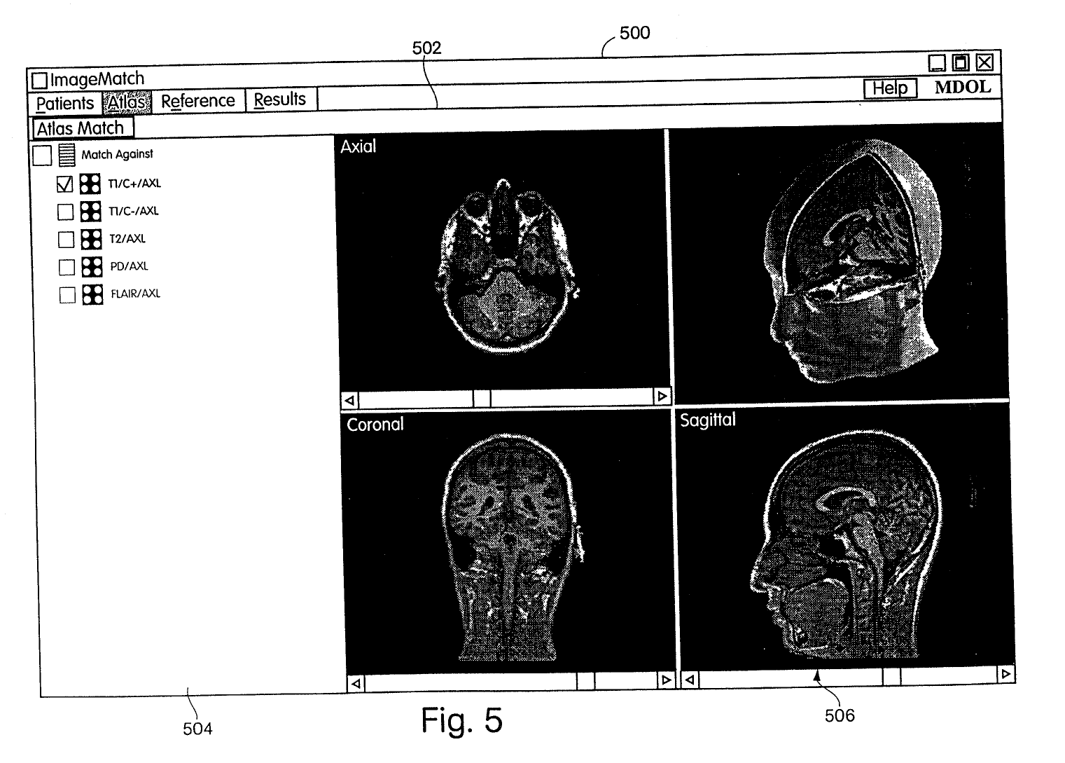

FIG. 5 shows an atlas workspace of a user interface. The atlas workspace 502 may include a view selection menu 504 and an atlas view space 506. The view selection menu 504 may be used to specify, for example, an anatomical region, an imaging modality, a pathology, or any other criteria useful for selecting a library of images.

The atlas view space 506 may provide views of subject matter selected in the view selection menu 504. For example, the atlas view space 506 may display an axial view, a coronal view, a sagittal view, and a three-dimensional cut-away view based upon images such as MR images. Each view within the atlas view space 506 may include one or more navigation tools. For example, the axial view, the coronal view, and the sagittal views provided as examples in FIG. 5 may include a scroll bar for navigating through different slices of an MR study. In a three-dimensional cut-away view, also shown as an example view in FIG. 5, each plane of the cut-away may be determined by one of the other views in the atlas view space 506. The three-dimensional view, or tri-plane view, may also be separately controlled to display planes of varying depth in each dimension. The three-dimensional view may be rotated about its axes using, for example, keyboard input from the client device or mouse-over movements within the three-dimensional view space. Labels may also be associated with regions within the atlas. Labels may then be automatically displayed within the views based upon mouse positioning within the view. Or a user may initiate a query for a label corresponding to a particular location.

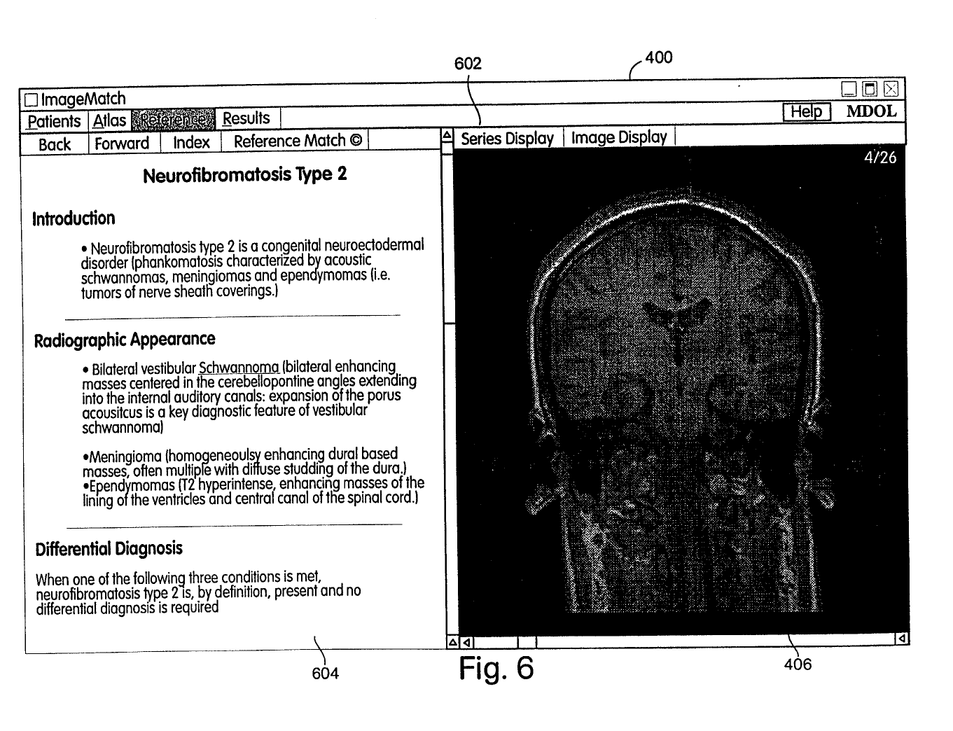

FIG. 6 shows a reference workspace of a user interface. The reference workspace 602 may include a text area 604 and an image area 606. The text area 604 may include, for example descriptive, educational, or diagnostic information for normal anatomy and abnormalities. Text within the text area 604 may be hyperlinked to other reference materials locally maintained for access through the reference workspace 602, or remotely accessible through the internetwork. The text area 604 may be accompanied by one or more buttons such as a back button for moving backward through the text, a forward button for moving forward through the text, and an index button for accessing and index or table of contents for reference materials available through the reference workspace 602. One or more scroll bars may also be provided for manually navigating through a section of reference text.

The image area 606 may display a series or a single image relating to text displayed in the text area 604. Navigational tools may be provided for user control of images displayed in the image area 606, such as a scroll bar for navigating through slices of an MR study. A matching button may be provided for matching a current image, e.g., an image accompanying the reference text, to other images and/or clinical data stored within the system.

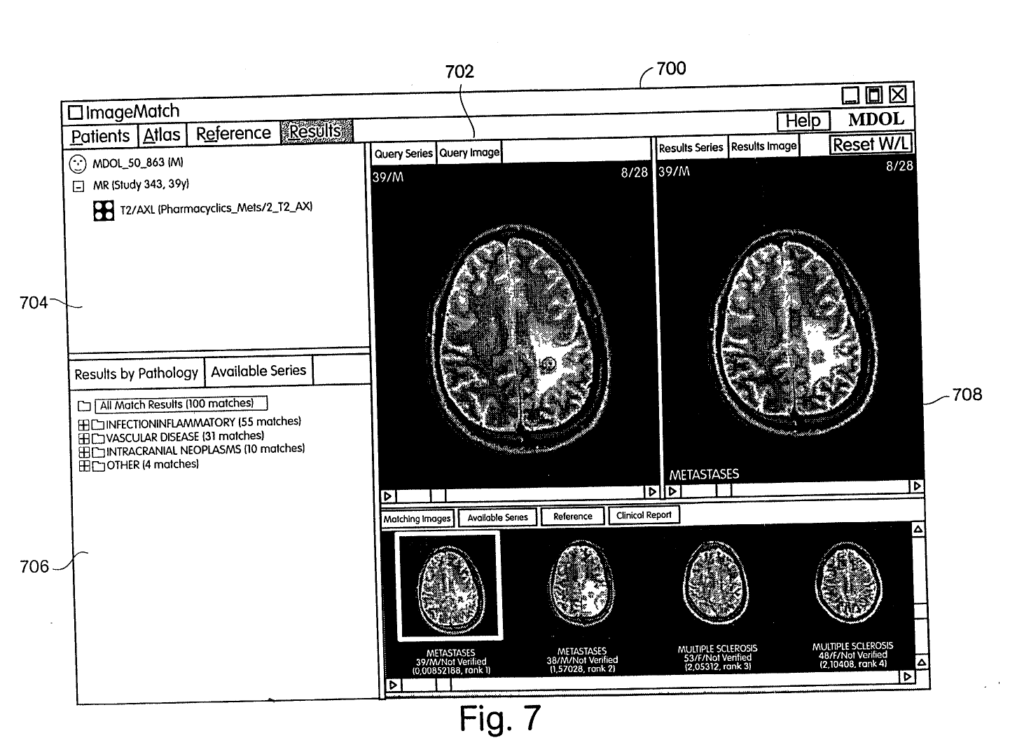

FIG. 7 shows a results workspace of a user interface. The results workspace 702 may include a query area 704, a results area 706, and an image area 708. The query area 704 may show one or more images for analysis through the user interface 400. The results area 706 may show one or more matches to the images, and/or associated clinical data, from the query area 704. The matches may be organized, such as by pathology or by queried database, and may be ranked according to a score derived from matching criteria. The image area 708 may display a query image associated with a subject listed in the query area 704, including any regions of interest identified within the image(s). The image area 708 may also display a result image, including one or more images and associated clinical data matched to the query image, such as through the matching techniques described below. The image area 708 may also display thumbnails of one or more result images along with any descriptive information, such as an associated pathology, a similarity ranking, a relative matching score, clinical data, and biographical data for an associated patient. A user may select one of the thumbnails for display as a result image.

In one aspect, the user interface 400 provides a platform for multi-modal matching or a multi-modal search engine. Each mode may provide a type of matching against a database of images and other information, which may be, for example, an image database containing images pre-processed for matching, as described in more detail below. Atlas matching may be provided, such as in the atlas workspace 502, in which images may be retrieved that match the position (in one, two, or three reference planes) being viewed in, or selected within, a three-dimensional object displayed within the atlas. Similarity matching may be provided, such as in the reference workspace 602, in which images may be retrieved that match the appearance of an image being viewed. Similarity may in this context be measured using matching and scoring techniques described below, or any other technique for evaluating similarity between images and image data. Diagnostic matching may be provided, such as in the results workspace 702, in which a diagnosis is generated for a query image. The diagnosis may include similar images retrieved from the database, and further include more than one diagnosis, scored according to comparison with images and diagnoses available in the database

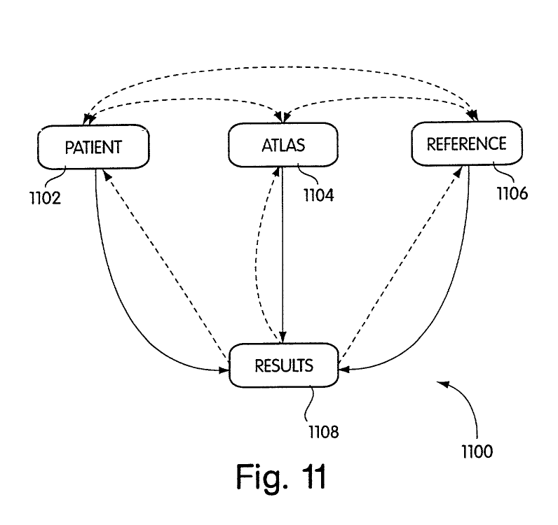

It will be appreciated that the above user interface may be used as a platform for workflow management in a clinical or other medical setting, as described below in more detail in reference to FIG. 11.

Having described an interface for using an image matching processing system in a medical context, a system for matching images is now described in further detail. The following matching techniques may be used in combination with the user interface described above in order to provide, for example, matched images in the results workspace 702.

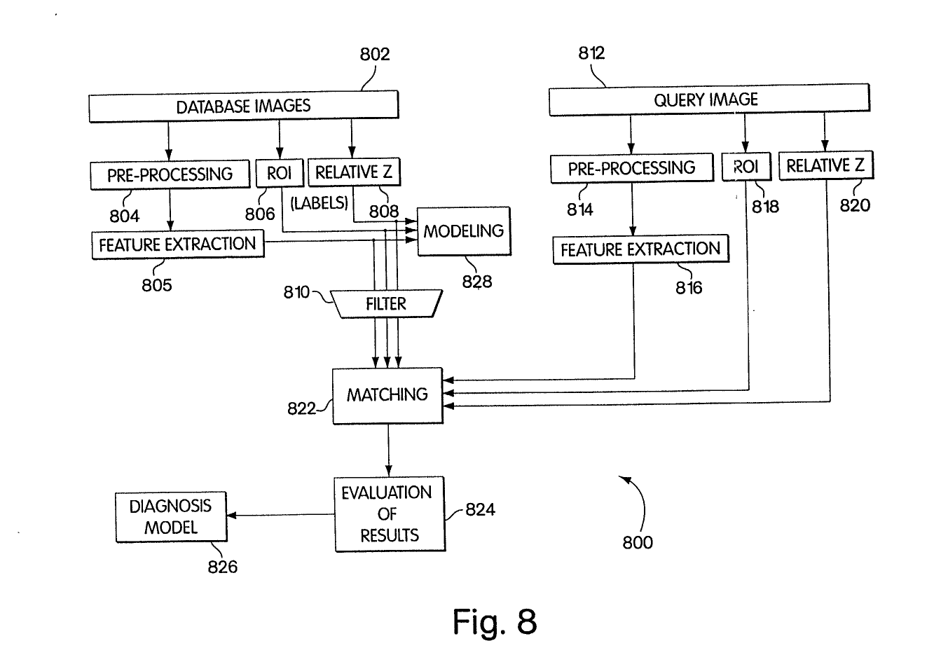

FIG. 8 is a flow chart showing a process 800 for processing images according to the systems described herein. The following example embodiment describes a method for processing neural radiology images obtained through magnetic resonance imaging. However, it will be appreciated that the techniques described herein may apply to a broad range of anatomical images obtained through a number of different imaging modalities, including x-ray images, computed tomography images, magnetic resonance imaging, ultrasound, and so forth. Any of these images, as well as non-medical images and other complex data or data structures, may be processed using the systems described herein.

In the systems described herein, there are periodic references to an x-direction, a y-direction, or a z-direction, along with related references to an x-position, a y-position, or a z-position and mathematically derived values such as an x-gradient or a y-gradient. It will be appreciated that an x-direction and a y-direction generally refer to two orthogonal axes in a planar image, and that a z-direction refers to a third axes perpendicular to the planar image. Other coordinate systems may be used with the systems described herein, such as the polar coordinates used for registration, which lie in the planar image.

It will be appreciated that, while the following description refers generally to cerebral magnetic resonance images, other image types are possible. For example, computerized tomography images may be used. Additionally, images may be taken from various anatomical regions, such as neck images, spine images, or musculo-skeletal images. All such image types are intended to fall within the scope of the systems described herein. Furthermore, the systems described herein may be extrapolated to full three-dimensional figures, as distinguished from the series of planar two-dimensional images typical of an MRI study.

As shown in step 802, database images may be provided. These may be, for example, an axial MR study including digitized images of each slice of the study. Additional data may be associated with each image, such as imaging details (e.g., pulse sequences such as T1, T2, FLAIR, and PD, or orientations such as axial, sagittal, or coronal), anatomy (e.g., brain, torso, arm, chest, etc.), patient data (e.g., age, height, weight, patient identification, gender, diagnosis, clinical reports, etc.), and any other data (e.g., physician, number of images, date, time) that may relate to the images. The additional data may be provided with each study, as in DICOM headers used for medical images, or may be added (manually or automatically) as supplemental information for each study.

As shown in step 804, each image may be pre-processed. This may generally include normalization, segmentation, and feature extraction, each of which will be discussed in more detail below.

Normalization may begin with mask extraction using any suitable boundary detection technique to obtain a mask of the outside perimeter of an imaged anatomical region. Within the mask area, grayscale intensity may be normalized for enclosed pixels. The mask image may then be rotated and registered to a global coordinate system. This may be, for example, a two-dimensional registration for each slice of an MR study, applying a non-rigid, global transformation, to an atlas derived from sample data, or to some predetermined geometry approximately descriptive of the images. One suitable geometry for cortical images is an ellipse. A number of mathematical techniques are known for performing non-rigid registration, including deformable model based registration, geometrical or other landmark based registration, voxel property based registration, and so forth. Transformations between registered and unregistered images may be affine, projective, or elastic. Any such technique may be used with the systems described herein, provided that (visual) features within different images may be adequately superimposed in the global coordinate system to allow discrimination of pathological variations, relative z-position, and any other information that might be usefully extracted from images and further processed using the other techniques described herein.

Once an image has been registered to a global coordinate system, it may be sampled in discrete sections within the transformed image space. In MR imaging, for example, an approximately polar coordinate system (e.g., elliptical) may be used for the global coordinate system, and tiles may be arranged for discrete sampling along axes of the global coordinate system. Arrangement of tiles within a polar, elliptical, or nearly elliptical coordinate system may be useful for MR images. In this manner, tiles may be arranged to coincide with regions of interest known to be associated with pathologies.

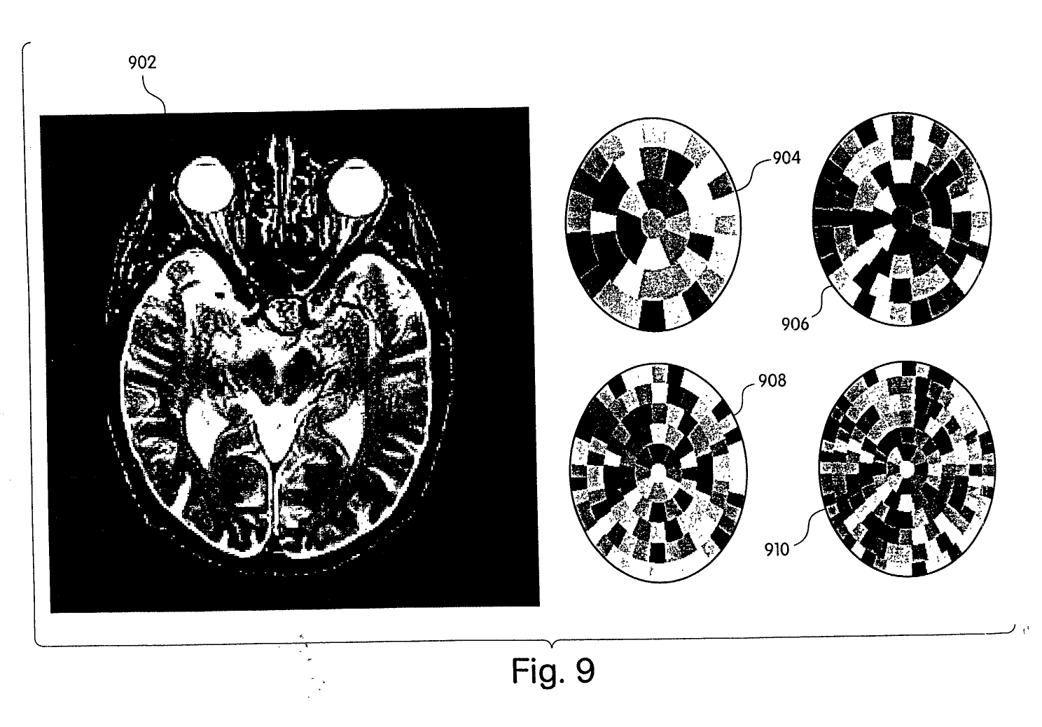

Referring briefly to FIG. 9, several possible arrangements of tiles for an MR image are shown. A slice of an MR study 902 may be masked, and the mask may be registered using a non-rigid transformation to an approximately elliptical shape. It will be appreciated that the transformed dimensions may be arbitrary, particularly where it is not used for visualization. However, suitable selection of a coordinate system for the transform may reduce computational costs, simplify registration, and map more effectively to the structure of the imaged subject, and implicitly, likely areas of diagnostic interest. One suitable transform is to the approximately elliptical shape shown in FIG. 9. The shape may then be tiled in any suitable manner. For example, a relatively coarse tiling 904 may use forty-nine tiles to cover the masked, registered image. Finer tiling 906, 908, 910 may be used where regions of interest have smaller dimensions. It will also be noted that tiles may be strategically arranged to coincide with likely regions of interest, such as to cover an area directly north (as oriented in FIG. 9) from the center, which corresponds to an indication of Pituitary Microadenoma at the depicted z-position.

Referring again to FIG. 8, image data in each tile may be sampled, and feature vectors may be extracted, as shown in step 805. Sampling may be, for example, an N×N matrix of grayscale values from within each tile. Feature vectors may include, for example, tile size, mean signal intensity (as normalized), standard deviation of signal intensity, mean edge magnitude, fraction of edge points above a threshold, mean x-direction gradient, mean y-direction gradient, mean absolute value of surface curvature, and mean absolute value of levelset curvature. In one embodiment, forty-nine tiles are used to cover the mask space, and ten feature vectors are calculated for each tile.

As shown in step 806, the database images may be segmented by, for example, identifying regions of interest within each image. This may be performed manually through, for example, a graphical user interface. Manual entry may be particularly useful where there is no a priori information about the database images and expected regions of interest. As will be described in further detail below, this may also be automated by modeling images with known regions of interest, and applying these models to new images to test for the presence of certain regions of interest. Regions of interest may be designated as points, as geometric shapes (e.g., square, triangle, circle, ellipse, trapezoid, etc.), or as hand-drawn or otherwise free-form open or closed curves.

As shown in step 808, the database images may be inspected to determine a relative z-position of each slice, such as of an MR study. This may be performed manually through, for example, a graphical user interface. Manual entry may be particularly useful where there is no a priori information about the database images and expected relative z-positions. As will be described in further detail below, this may also be automated by modeling images with known relative z-positions, and applying these models to new images to determine a relative z-position.

More generally, while steps 806 and 808 depict labels of images for regions of interest and relative z-position, any labels may be similarly added to images, or regions of images, to correspond to observed characteristics (e.g., diagnosis, diagnostic significance of a region of interest, unusual characteristics, clinical indications associated with a patient, and so forth) or other global attributes of the images. These labels may be added manually to a database by inspecting images and associated data, and adding any suitable or desired labels. For example, a qualified physician may examine images and other patient data, and label images or locations therein as indicative of a specific pathology. Models may also be constructed from manually labeled images to automate subsequent labeling of data. It will also be appreciated that any known computational techniques may be applied for automated labeling, without regard to computational cost, without affecting subsequent matching and modeling steps described herein.

As depicted in step 810, a filter may be provided for one or more databases of images that have been pre-processed as described above. The filter may receive data relating to the query image, as described in more detail below, and may prepare a subset of data from the database of images that is to be used for subsequent matching. For example, the database of images may be filtered based upon pathologies, orientation and sequence, z-location range, and region of interest. Filtering by pathology may exclude all normal image cases (i.e., those not exhibiting any pathology), or may exclude all images not exhibit a specified pathology where, for example, a particular pathology is being tested for. Orientation and sequence filtering may remove images obtained, for example, on a different axis from the query image, or using a different imaging modality. For example, MR imaging may employ several different pulse types, sequences, and so forth, and each type may produce visually different results for the same region, thus rendering image-based comparisons meaningless, or at least less useful. Filtering may also be performed by z-location using, for example, the derived or manually added z-position data provided in step 808. Filtering may also be performed by region of interest, using, for example, the derived or manually added region of interest data provided in step 806. Using these filters, subsequent matching steps may be performed on a subset of the complete database of images.

As shown in step 812, a query image may be provided. This may be, for example, an axial MR study including digitized images of one or more slice of a study. Additional data may be associated with each query image, such as imaging details (e.g., pulse sequences such as T1, T2, FLAIR, and PD, or orientations such as axial, sagittal, or coronal), anatomy (e.g., brain, torso, arm, chest, etc.), patient data (e.g., age, height, weight, patient identification, gender, diagnosis, etc.), and any other data (e.g., physician, number of images, date, time) that may relate to the images. The additional data may be provided with each query image, as in DICOM headers used for medical images, or may be added (manually or automatically) as supplemental information for each query image.

As shown in step 814, each query image may be pre-processed. This may generally include normalization, segmentation, and feature extraction, each is discussed above with reference to database images. Further steps such as feature extraction shown in step 816, region of interest determination 818, relative z-position 820, and any other labeling with observed characteristics or other global attributes may be performed on the query image(s). Regions of interest, for example, may be identified by a user with a single point-and-click mouse operation at a client device where the query image is being reviewed. It will be appreciated that, in a client/server architecture such as those discussed above, a browser-based or application-based client may perform steps 814-820 at a client device, with matching performed at a remote location accessible through the internetwork. Matched images may then be returned to the client device for review by a user at that location. Other arrangements are possible such as receiving unprocessed query images at a workstation that has the database of images, filter, and so forth locally available. Where the filtered database becomes sufficiently small, the output of the filter may also be provided to a client device for local matching at the client device. Full images may then be retrieved for review at the client device on an ad hoc basis, depending on results of a match performed at the client device. All such variations are intended to fall within the scope of the systems described herein.

As shown in step 822, matching may be performed between the query image and one or more images from the database of images. More particularly, a pre-processed query image and accompanying labels may be compared to a filtered set of pre-processed images from the database of images. As noted above, the labels for the query image may be used, in part, to filter data from the database of images. In one embodiment, a matching algorithm may receive from the filtering step 810 any features extracted for images that satisfy filter constraints. These feature vectors may be compared to one or more corresponding feature vectors from the query image. A scoring mechanism for this process may be chosen from a wide spectrum of procedures, such as a simple two-norm, inner product, or correlation, or other similarity measure. Elements of each feature vector may be weighted according to an empirical or formulaic evaluation of results. Results of the scoring process may be further processed, such as by removing results below a certain matching score threshold, and by sorting according to score, pathology, and so forth.

In certain cases, the matching may be improved by appropriately weighting one or more feature vectors. For example, a Mahalanobis distance may be applied to normalize weights for feature vectors used in the model. In application, matching may be performed by computing a weighted norm of a difference between feature vectors for a query image and each image that the query image is tested against. Such a weighting may be derived from information contained within the database and may be a function of various quantities such as spatial location, known pathology, or imaging parameters. The weighted norm may be a Mahalanobis distance derived from a covariance matrix of database feature vectors. More generally, the weighted norm may be any function of one or more labels or other data, including spatial position, pathology, and so forth.

As shown in step 824, results may be evaluated. This may be conducted by a user through, for example, the user interface described above with reference to FIGS. 4-7. Once a match has been made, any or all of the data relating to the matched image may be retrieved and reviewed by a user, including a full MR study, associated clinical data, biographical patient data, labels, and so forth. The evaluation may include references to an atlas, reference materials, and other data that may be available through the user interface described above. Furthermore, any conclusions or other findings may be associated with the query image and stored along with the query image in a suitable database.

At the conclusion of the evaluation, findings may be provided to a diagnosis model as shown in step 826. For example, a neural radiologist may review a query image, along with all associated clinical and other data. The radiologist may further review matched images and associated data obtained through the matching process of step 822. If the radiologist concludes that a particular diagnosis is appropriate, this indication may be provided to a modeling system along with the query image, associated data, and any other data derived therefrom, such as feature vectors, regions of interest, relative z-positions, and any other labels or other attributes. Query images stored in this fashion may provide, for example, a ground-truth database of diagnosed cases from which diagnostic models may be obtained using techniques described below. A diagnostic model may be used to automate diagnosis of subsequent query images, or to recommend one or more diagnoses to a qualified physician.

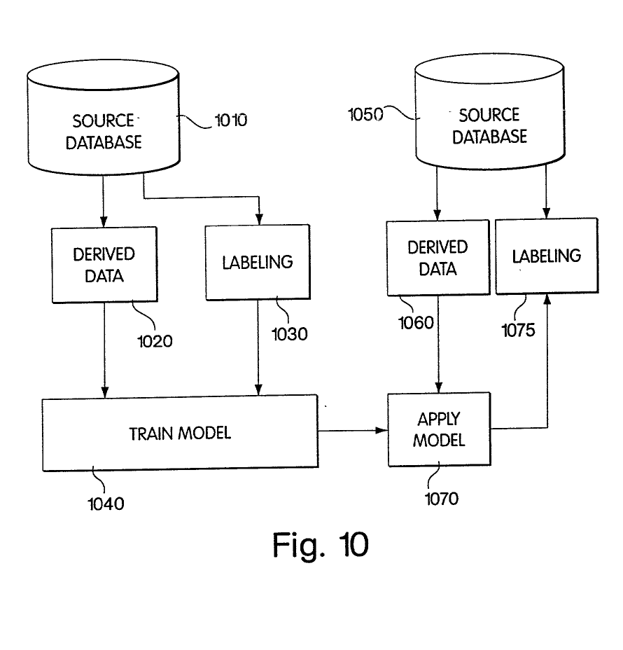

As shown in step 828 other modeling may be performed. This modeling may be performed on any combination of image data from the database of images, pre-processed image data, including feature vectors, regions of interest, relative z-position, and any other labels, as well as other data associated with the images as described above. Modeling may generally include training a model to associate one or more inputs with one or more outputs. For example, feature vectors may be associated with a label, such as relative z-position, or with a diagnosis of a pathology. Techniques for modeling include, for example, regression analysis with regression coefficients determined through least squares or partial least squares, neural networks, fuzzy logic, and so forth. Regression modeling, for example, has been usefully applied to feature vectors for MR images to identify z-location, to note the presence of contrast enhancing agents, and to discriminate between Ti and MRA images. These models may be applied, for example, to automate labeling processes and to label new databases, or to check the accuracy of labels for databases that already include the label information. Modeling is discussed in further detail with reference to FIG. 10.

It will be appreciated that the above process 800, may be realized in hardware, software, or some combination of these. The process 800 may be realized in one or more microprocessors, microcontrollers, embedded microcontrollers, programmable digital signal processors or other programmable device, along with internal and/or external memory such as read-only memory, programmable read-only memory, electronically erasable programmable read-only memory, random access memory, dynamic random access memory, double data rate random access memory, Rambus direct random access memory, flash memory, or any other volatile or non-volatile memory for storing program instructions, program data, and program output or other intermediate or final results. The process 500 and the shape processor 200 may also, or instead, include an application specific integrated circuit, a programmable gate array, programmable array logic, or any other device that may be configured to process electronic signals.

Any combination of the above circuits and components, whether packaged discretely, as a chip, as a chipset, or as a die, may be suitably adapted to use with the systems described herein. It will further be appreciated that the above process 800 may be realized as computer executable code created using a structured programming language such as C, an object-oriented programming language such as C++ or Java, or any other high-level or low-level programming language that may be compiled or interpreted to run on one of the above devices, as well as heterogeneous combinations of processors, processor architectures, or combinations of different hardware and software. The process 800 may be deployed using software technologies or development environments including a mix of software languages, such as Microsoft IIS, Active Server Pages, Java, C++, Oracle databases, SQL, and so forth. In addition, it will be appreciated that, as where noted above, certain steps to the process 800 may be realized in part through input from a human user, such as where initialization of a model is to be based on diagnoses from a trained radiologist.

FIG. 10 is a flowchart of a process for organizing databases. While the following example details a technique for organizing cerebral MRI images using statistical techniques to develop regression coefficients, it will be appreciated that the general approach described herein may have general application to organization and searching of large databases, and in particular to databases of medical images. For example, a model that relates feature vectors to a relative z-position may be used to automate labeling of relative z-positions for slices of an MR study. Or, a model that relates regions of interest and feature vectors to a diagnosis may be used as a diagnostic tool. These and other applications are intended to fall within the scope of the system described herein. As an example, text-base modeling may be performed, in which textual patient reports may be analyzed and modeled for labeling by diagnosis or other criteria.

A source database 1010 may be used to obtain derived data, as shown in step 1020. The source database 1010 may be for example, a database of MR studies, along with clinical and other patient data. Derived data may be data obtained through any predetermined function. For example, the feature vector extraction described above with reference to FIG. 8 produces derived data in the form of feature vectors for an image, or for a region of an image. The derived data may optionally be stored in the source database 1010 along with the images.

As shown in step 1030, images may then be labeled. This may include manual labeling of regions of interest or z-positions, as described above. This may also include identification of data already associated with images in the source database 1010, such as a diagnosis, or patient data such as age, sex, height, weight, and so forth.

As shown in step 1040, labels obtained in step 1030 and derived data obtained in step 1020 may be applied to a model, such as a regression model or a neural network model, and coefficients may be obtained that relate inputs (e.g., derived data) to outputs (e.g., labels). The model may employ, for example, linear regression, or any other statistical learning methodology. The resulting trained model may be applied to a target database 1050 as described below. The model may be adapted to different types of outputs. For example, a model with a scalar output may be used for a one-dimensional result, such as z-position. Such a model may also be applied to binary inquiries. For example, where a model is constructed to test for the presence of a contrast agent, a larger value may correspond to a contrast agent while a smaller value may correspond to no contrast agent. Where more information is required, such as an x-location and y-location (or angle and distance) for a region of interests, a multi-dimensional output may be appropriate.

As shown in step 1060, data may be derived from the target database 1050. While any technique may be used to derive data from the target database 1050 in step 1060, as well as to derive data from the source database 1010 in step 1020, the same technique should be used in both cases so that the model trained in step 1040 will yield meaningful results. As shown in step 1070, the model may be applied to the data derived from the target database 1050, and the output of the model may be used to label data from which the derived data was obtained. This procedure may be useful, for example to prepare a new database of MR images for use with the image processing systems described above. More particularly, operations such as manual data entry of relative z-positions for new images may be replaced with automated determination of relative z-position, and images automatically labeled with z-positions in this manner may be added to a source database for image matching.

A model trained as described above may also, or instead, be used to test the integrity of pre-existing labels in databases. Following the example above, a database may already include relative z-positions for images in the database. The model may be applied as above to independently determine relative z-positions. The relative z-positions may then be compared in a number of ways. For example, the model output may be used to replace preexisting labels, or may be used to provide labels for any images missing this information. The results may be reported statistically as deviations from expected results. Or the results may be used to exclude from subsequent searches those images with pre-existing labels significantly different from a corresponding model output. The significance of the difference may be determined using an absolute or relative threshold for excluding certain database records.

The above approach to modeling databases may be applied in a data mining system. For example, a physician may search image databases for images matching a particular patient by applying the modeling techniques above. The physician may, at the same time search text references, clinical histories, and any other data from other data sources, and gather search results into a library of possibly relevant materials. Further, a number of different models may be used to assist in searching databases having different data types. Image data may include, for example, neural CT scans, neural MR studies, and other image types. Modeling may also be applicable to other data types, such as text-based patient histories, provided that the data can be labeled with one or more observed characteristics, and processed into some type of derivative data for which a model can be accurately trained to generate the labels. There is accordingly described herein a data mining system in which one or more target databases can be modeled and queried, either alone or in conjunction with other searches.

FIG. 11 shows a state diagram for a workflow management system. The workflow management system 1100 may be formed around several states of a user interface, such as a patient state 1102, an atlas state 1104, a reference state 1106, and a results state 1108. As indicated in FIG. 11 by arrows interconnecting these states, a user may navigate between states in any suitable manner while working to conclude with a diagnosis for a patient.

For example, the system may be initiated in the patient state 1102, where a radiologist may receive an MR study for review in a user interface, such as the patient workspace 402 of FIG. 4. The radiologist may navigate to the atlas state 1104, where a workspace such as the atlas workspace 502 of FIG. 5 may be used as a guide to visual review of an anatomical area imaged by the MR study. The radiologist may navigate to the reference state 1106, where a workspace such as the reference workspace 602 of FIG. 6 may be used by the radiologist to investigate any potential diagnoses. Each state may be selected by, for example, selecting a window tab in a Windows environment, or using a navigation panel in a Web browser.

The results state 1108, which may be presented in a workspace such as the results workspace 702 of FIG. 7, may be reviewed by the radiologist, or other clinician, as a farther diagnostic aid. The results state 1108 may be reached using navigation methods noted above, where image matches and associated data, such as clinical data, from a previous search may be reviewed. The results state 1108 may also be reached through an explicit search instruction provided in one of the other states, e.g., the patient state 1102, the atlas state 1104, or the reference state 1106. A search from the patient state 1102 may be for images in an image database that match image data for a patient currently under review, and may include, for example, a user-specified region of interest. A search from the atlas state 1104 may be for all images corresponding to a user-specified location within an atlas displayed in the atlas workspace 502. A search from the reference state 1106 may be a search upon a user provided criterion, such as a pathology, a location, and so forth.

A radiologist using the system for diagnosis may traverse the states as desired, moving for example, from the results state 1108 to the reference state 1106 for further information about one or more pathologies, until a conclusion is reached concerning a current MR study. The MR study may then be dispatched from the system, along with any findings, and additional MR studies may be retrieved for analysis. Where MR studies are provided directly in digital form, they may be pre-processed as described below and provided to the workflow management platform for review by a clinician in a fully paper-less system.

It will be appreciated that other applications for the above systems are possible. For example, where all images are registered to a global coordinate system as described above, a spatial probability map may be created for a pathology. That is, each region of interest in an image associated with the pathology may be combined using known techniques, and spatial distributions of the regions of interest combined so that the likelihood of a region of interest (such as a lesion) appearing in a certain location for a certain pathology may be determined. Following this approach through the entire coordinate system, a complete spatial probability map may be derived for a particular pathology. The map would reveal, for each location within the coordinate system, the probability of a lesion appearing for the pathology. Such a map may be used as a diagnostic aid, or as an aid to identifying regions of interest in manual or automated labeling systems such as those described above.

It will further be appreciated that a number of different architectures may be based upon the system described herein. For example, a client providing the user interface and query image pre-processing described herein may be licensed for remote use through a Web browser plug-in, remote use through an application service provider, or remote use through a proprietary local client. The software and/or hardware system may, instead, be sold or licensed for use in its entirety as a clinical tool for use on a local or corporate area network of a hospital.

The systems described herein may be extended to operate as a just-in-time search engine. One of the most valuable resources in a medical care environment is time. A typical database system usually involves the generation and submission of a user-defined query that is processed by the database while the user waits for the results. This commonly used model is appropriate in situations where the query and the results are relatively small in size to transmit, and the processing of the query occurs quickly. However, in the medical domain, there are numerous delays that can occur between the time when the query is initially submitted and the time when the results have been received.

The query consists, in part, of the results of patient tests, and may include all forms of digital information. Unlike text-only queries that are relatively small in size, three-dimensional medical imagery is slow to transmit over existing networks, and can be a major source of delay. While the query generally consists of a small number of images, the database may return a large number (20 or more) of relevant images as results, causing significant delay in receipt of the entire reference set. The database may also return other information, such as three-dimensional models of the anatomical structures present in the reference images, or case-independent information mined from online medical encyclopedias. Together, all of the multimedia information, such as text, imagery, audio, video, contained in the returned knowledge base can account for a substantial amount of data and thus requires time to transfer over the networks.

In addition to limitations in the network bandwidth, the analysis of the query also contributes to the delay. In order to determine which subset of the database is relevant to the patient's condition, portions of the query are matched to the elements of the database to find similarities. For example, an undiagnosed magnetic resonance (MR) image of a patient may be compared to MR images in the database that are tagged with diagnoses. A certain diagnosis of the query image is greatly supported when multiple images from the database with the same diagnosis all show a high degree of similarity to the query image. One practical challenge in this use of this matching system lies in the fact that the analysis time may not be constant for all cases. Pathology present in some imagery may be rapidly identifiable, while other cases may require more resources to perform sufficient analysis. When longer analysis time is required, it is unreasonable for the user to be idle, waiting for the results.

While many database-query systems consist of a one-stage query-response paradigm, a more general, and potentially more powerful approach involves a hierarchical, multi-stage information gathering protocol. For example, when an image matching system generates a list of similar medical images with corresponding diagnoses, the database system may in turn query other available resources based on the matched diagnoses and consolidate multimedia information to create a unified presentation of the results. While this multistage information mining process is a useful diagnostic aid, it may require sufficient time that if the query is submitted at the time of evaluation, the physician may not be able to wait for the results.

The delay inherent to remote medical information gathering may seem too large for practical use by physicians. However, a mining framework other than the standard method of submitting the query and waiting for the result provides a solution. In practice, there is generally a delay between the time in which medical tests are performed and when a physician evaluates the results. For example, a radiologist technician may acquire a set of medical images of a patient hours before the radiologist analyzes the imagery. If the medical image is sent as a query to the database system at the time of acquisition, the delay between acquisition and evaluation allows for ample time for the query image to be sent to the remote database, for identification of relevant reference images from the database, and for transmission of the reference images back to the remote location.

Generally, the amount of time between the receipt and evaluation of test results varies from case to case. In non-emergency situations, the physician generally does not review the patient's medical imagery or test results as soon as they become available. Therefore, the existing delay between the acquisition and medical evaluation can be used effectively for transmission of data and extensive processing of the query, as well as for scheduling the transmission with other, more urgent requests.

In one embodiment of the present invention, the mining system consists of a gateway medical station that collects key input data for a patient from multiple sites within the medical facility, immediately upon availability of the data. The gateway also plans, schedules, and initiates all remote queries to most effectively use the resources available and to balance the needs of the individuals who submitted the queries. The key input data collected by the gateway usually consists of medical test results in the form of digitally encoded textual and/or graphical information. For example, medical images, such as magnetic resonance (MR) or computed tomography (CT) of a patient are acquired and stored digitally in the radiology department, while the patient's blood tests results are stored on the blood lab computer system. The gateway medical station would collect this and other patient-specific digital information as it is generated.

In addition to collecting the patient-specific information, the gateway also processes and organizes it into a set of database queries. The queries are then submitted to the relevant remote medical databases in order to retrieve additional useful resources for presentation to the physician at the time of evaluation. In essence, the “Just in time” system uses the available medical information to anticipate various retrieval requests that may be submitted by the physician at a later time, during the case evaluation. The choice of what information to mine depends on information contained in the patient's records and the types of medical images acquired and tests performed. The technicians running the various diagnostic procedures can choose from a set of a priori mining procedures and can also submit requests for specific information. By processing, analyzing, and fetching relevant information immediately, the system leverages the elapsed time that generally occurs between the acquisition of the medical tests and evaluation of the results.

The scheduling algorithm incorporated into the data mining system balances the needs of the users with the available resources in a variety of ways. Given the potentially large number of simultaneous requests and large amount of data being transferred, the requests cannot simply be processed in one chunk as they arrive, since the urgency of information requests will vary significantly from case to case. In addition to test results, the gateway medical station also receives as input the expected time that the results will need to be reviewed by the physician. These parameters of the mining process are set at the time of acquisition either by the technician or doctor, in order to encode the urgency of the request.

With this information, the gateway prioritizes the queries and schedules the submission of the queries, taking into account the expected length of time for transmission and processing of the query, as well as for receipt of the results. The information from any one patient may also be transformed into multiple queries submitted to multiple databases or preempt more urgent queries, while still ensuring receipt of the necessary information in the allotted amount of time. The scheduling system also takes into account the fact that information received from one database may be used as part of a future query to another database, requiring ample time to perform the multi-stage data mining.

In order to transmit information in the correct form to the appropriate database systems, the gateway stores information about the various remote sites, such as the type of data in the remote database, the types of queries allowed, and the service level agreement (SLA) between the local and remote institutions. The SLA is a contract that determines the cost, response time, and resource constraints when a query is sent to a given search engine. For a standard type of query, the remote site defines the cost and how long before the results are returned. In urgent situations, the medical facility may need a response in less time than guaranteed by the basic terms of the SLA. Often the SLA includes the option of a rush order, at increased expense, guaranteeing the result in less time. This is most practical when the physician or radiologist includes with the data a constraint that the results are needed by a certain time (e.g. before a scheduled surgery). In general, the SLA provides a list of response times as a function of query types and expense that the gateway processes and uses as part of the prioritizing and scheduling algorithm to mine the needed remote information. The gateway scheduling system needs to take into account not only the information provided in the SLA, but also the policies as defined by the medical facility for transmission of such queries. For example, the medical facility may limit the expenditure on certain types of queries, so as to not have every request marked as urgent.

During times when there is a large load on the mining coordination system and external networks or when available time is severely limited, the gateway system will prioritize which data and query types are most relevant to transmit given the finite resources. This process includes organization of the data in a way to most effectively satisfy all the supplied constraints while accruing the least cost from the remove servers. Organization of the data involves stratification of the various requests, and in some cases, breaking apart the data into packets, and interleaving the packets as necessary to stream the information to allow remote processing to begin as soon as possible. This data organizing stem is also important since a remote site may limit the amount of data transmitted to its site in a given amount of time so as not to overload its own network pipeline, as specified in the SLA.

Once all of the query results for a given case have been received by the gateway mining system, the gateway then dispatches the information back to the appropriate local medical data repositories. The mined information will then be available when the physician is prepared to analyze the patient's medical data for diagnosis and treatment purposes. In contrast with prior art, this method provides timely access to practically unlimited number of medical knowledge bases and, at the same time, it provides the characteristics of real time medical information mining services.

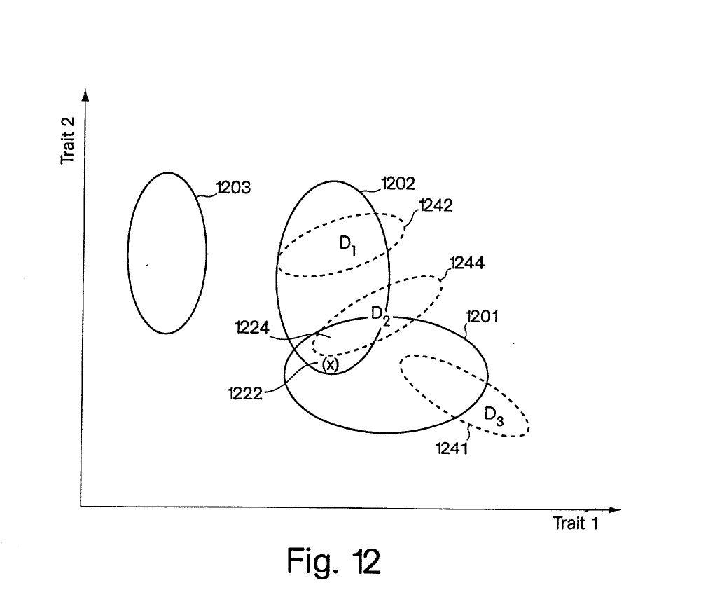

The systems described herein may also be applied to create self-forming groups based upon common interests, such as medical conditions. Referring now also to FIG. 12, a database may include medical data of diagnosed cases which can be linked to a medical diagnosis and/or categorized in other ways known in the art. The medical data span a multidimensional space, including, for example, such parameters, or traits, as age, gender, patient/family history, test results (blood pressure, cholesterol, other essays), as well as vector quantities (EKG) and possibly also images. FIG. 12 is a two-dimensional diagram of two exemplary traits, trait 1 (x-axis), trait 2 (y-axis) spanned by the database. Trait 1 may be a blood pressure reading and trait 2 an EKG characteristic signal. A point or area in the diagram will have a likelihood of one or more medical diagnoses associated with it. Schematically indicated in FIG. 12 are the mean values D₁, D₂ and D₃ for three different exemplary diagnoses as well as elliptical envelopes 1242, 1244, 1241 surrounding each mean value D₁, D₂ and D₃ and representing an accepted confidence limit of value pairs (trait 1, trait 2) for a respective diagnosis D₁, D₂ and D₃. The mean positions D₁, D₂ and D₃ of each cluster is unique to the particular diagnosis.

A first client may provide a server with exemplary patient data 1201 having a range for traits 1 and 2. The patient data 1201 overlap with both the envelope 1244 for diagnosis D₂ and with envelope 1241 for diagnosis D₃. In other words, the client could have either the disease D₂ or the disease D₃. Likewise, a second client may provide the server with exemplary patient data having a range 1202 for the same traits 1 and 2. The patient data 1202 overlap with both the envelope 1242 for diagnosis D1 and with envelope 1244 for diagnosis D₂. In other words, the second client could have either the disease D₁ or the disease D₂. Additional traits 3, . . . , N may help discriminate against the various possibilities for D_{i.}

As also seen from FIG. 12, the ranges 1201 and 1202 overlap in overlap region 1222. Moreover, a significant portion of overlap region 1222 also overlaps with the likelihood region for disease D₂ as defined by envelope 1244. The system will hence group clients into a self-assembled group and associate them with the diagnosis D₂. In other words, the aforedescribed method will automatically cluster the patients into finely granulated interest groups, in turn allowing users to easily obtain the richest information available concerning the details of their condition. Thus, the clustering of individuals is done objectively based on patients matching similar cases in the database, instead of by using subjective criteria.

A quantitative measure for the similarity or correspondence between data and/or data sets can be based on the Mahalanobis distance computed in a Mahalanobis metric. The Mahalanobis distance is a very useful way of determining the “similarity” of a set of values from an “unknown” sample to a set of values measured from a collection of “known” or reference data. Since the Mahalanobis distance is measured in units of standard deviations 110 from the mean of the reference data, the reported matching values give a statistical measure of how well the unknown sample matches (or does not match) the reference data.

Visual inspection, like the diagram of FIG. 12, is usually not a viable method for actual discriminant analysis applications. An Euclidean distance method (“least-square-fit”) does not take into account the variability of the values in the different dimensions, and is therefore not an optimum discriminant algorithm. The Mahalanobis distance, on the other hand, does take the sample variability into account. Instead of treating all values equally when calculating the distance from the mean point, it weights the differences by the range of variability in the direction of the sample point. Mahalanobis distances look at not only variations (variance) between the responses for the same trait, but also at the inter-trait variations (co-variance). The Mahalanobis group defines a multi-dimensional space whose boundaries determine the range of variation that are acceptable for unknown samples to be classified as members and hence admitted to the respective group.

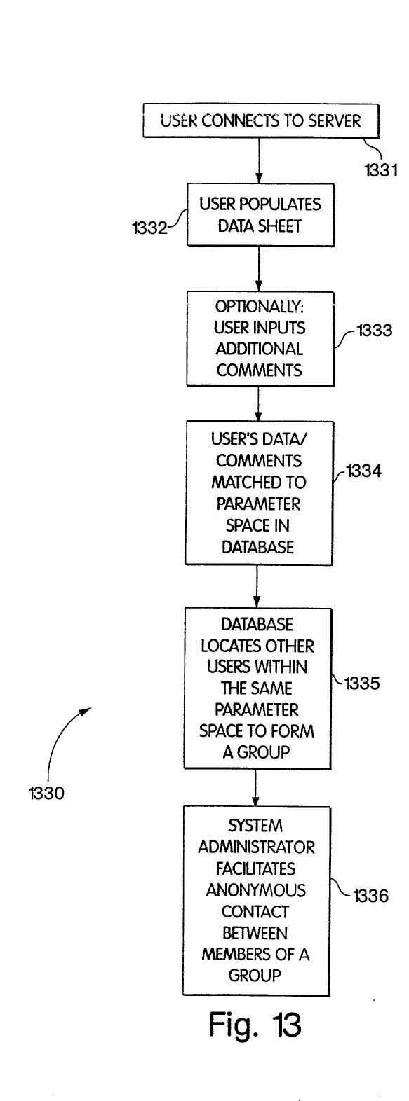

Referring now to FIG. 13, in an exemplary process 1330 for forming/joining a self-assembled group and communicating anonymously with other members of the group, a user/patient connects to the server with the medical database, step 1331, and populates a datasheet with the patient's data, step 1332. As mentioned above, populating the data sheet is not limited to entering text information. In many cases, the patient will respond to structured database queries (e.g., “What is your blood pressure?”, “Have you experienced dizziness?”, etc.); however, the patient may also wish to enter data or additional information in form of a comment, wherein the comment may not find a matching entry in the database, step 1333. Such comment may be valuable and may lead to a future modification and/or addition to a “labeled” database entry in the event that more patients enter similar comments. One example may be hitherto unknown side effects in a clinical or drug study.

In the database, the data/comments entered by the patient are mapped onto the data of diagnosed or “labeled” cases, step 1334, and other users/patients mapped onto the same data of diagnosed cases are identified, step 1335. At this point, the patient who entered the data/comments becomes a member of the same group and shares the diagnosis. The clients that are part of the group may have logged on anonymously or may have identified themselves to the server. In any event, the system is designed so that a client's identity is not revealed to another client, for example, by assigning an ID to the clients. Having assigned a client ID, the server can now facilitate anonymous contact between clients that belong to the same group, step 1336.

Anonymous communication can be established between clients of a group via an anonymous link, for example, through email addresses that are never associated with the user's identity or alternatively via a telephone patching system, where a third part establishes a one-time telephone connection without either of the two parties having any personal information about the other. When establishing anonymous email communication, email originating from one of the users can be sent to a third party with a unique, coded identifier corresponding to the communication path between the two parties. The third party forwards the email messages appropriately, and also allows either party to break the link at any time, in which the coded identifier becomes invalid, and all further communication between the two parties is terminated. Thus, the grouping communication system allows users to have open discussions with exchange of medical information and development of support networks, while still having the security that the identities of the users are protected and that users are in control of the email they receive. The anonymous link can, in fact, be designed in such a way that the service providers themselves (i.e., the database, server and/or search engine), have no knowledge of true user identities. By storing encrypted markers associated with anonymous email accounts, but never connected to the identities of the individuals, the server assures that anonymity is maintained. When an anonymous communication channel is requested between two members of the same group, the system can, for example, establish the connection by passing the anonymous email addresses to a re-mailer.

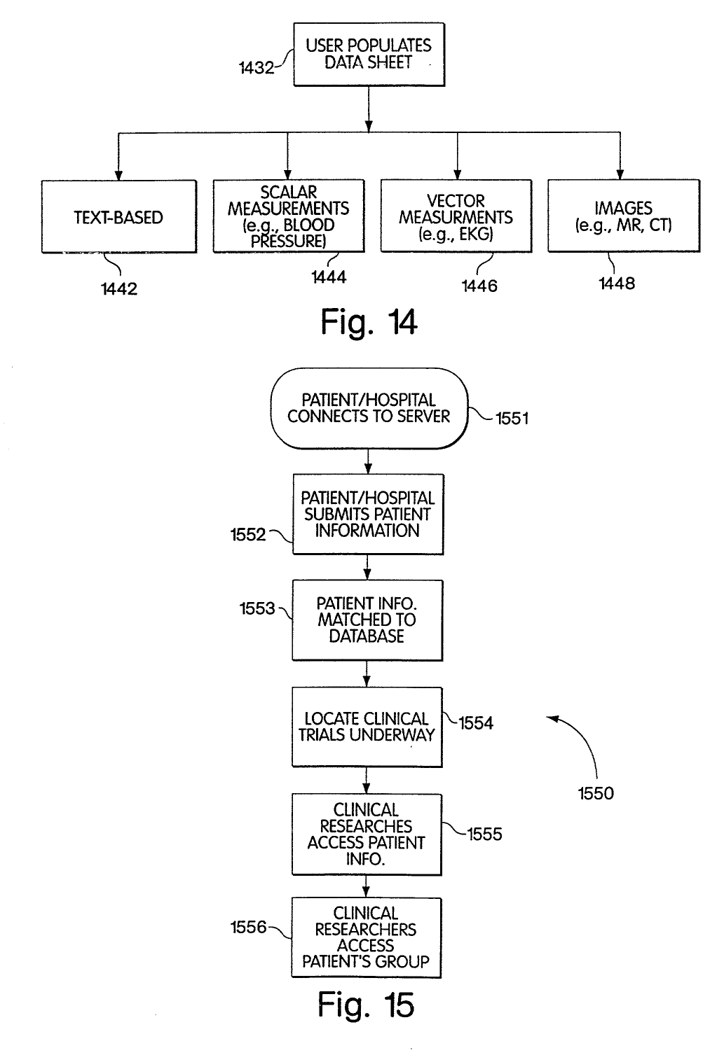

As indicated in FIG. 14, the database entries and patient data entries 1432 do not only include text entries 1442 containing medical information, but can also accommodate full multimedia matching of any modality of available data. Similar to a physician combining the available information when assessing the similarity of two cases, the automatic matching system can use a combination of text 1442 (e.g. patient history), scalar measurements 1444 (e.g. blood pressure), vector quantities 1446 (e.g. EKG), and images 1448 (e.g. MR/CT).

Patient to patient support group development is one of the major types of communication channels facilitated by the automatic clustering techniques of the present invention. There are a variety of ways of structuring the actual initiation of communication between individuals. In one embodiment, patients whose information is in the database are never contacted by patients who enter their own information. When a patient enters his or her data and asks for a search, at the end of the process (or at some other convenient time, the search engine will ask the client two questions:

1. Would the client like to initiate communication with people who have similar matches to his (her) own information in a confidential manner, and

2. Would the client be willing to be contacted by others in a like confidential manner in order to discuss his (her) case with someone else.

Within a short period, a large number of users with varying diagnoses and assigned to different groups may have agreed to participate in an inter-person communication. The search engine and related software would keep track of the case numbers matching a specific group and would also keep track of the case numbers in the database for each client who agreed to communicate anonymously. In this way, whenever a new client indicates that he/she would like to communicate with people being diagnosed with a similar disease, the software would search all clients who agreed to communicate with others to see if any of these other people's medical information matched the same cases in our database as the new client. The communication would be useful to the clients because each could share experiences with various decisions, outcomes, treatments, etc. The initial communication with the server would be anonymous via email, keeping names and identities separate until both parties mutually agree to identify themselves.

The described system and method may also allow patients to communicate with others having similar conditions, but without an associated diagnosis. In this case, like in the previously discussed case, an anonymous communication link 16 can be established between a patient submitting a query and a patient whose medical information exists in the reference database. The process used throughout should not offend a participating institution that supplied the database, should not violate confidentiality/privacy issues, and should not harm in any way the patient or family of the patient whose image was matched to the client, since the patient may not be aware of that his/her image was a part of the medical information search engine. As in the example above, a patient would indicate after using the search engine that he/she would like to communicate with a matching person from the database or the person's family. The trusted proprietors of the database would then send the request to the institution from where the image was originally received, using the encoded patient ID number, matching it to the list that is located in the computer bank of the sending institution. The sending institution would then search for the original patient's name, address, and email if available, and would then either send a personalized form letter or e-mail to the patient or family. This letter can originate from the treating institution and explain that the treating institution is involved in this research. The patient or family will be told that the patient's medical image was included anonymously in a database of information. The letter or e-mail will explain briefly the purpose of the search engine and that an individual who used the system would like to contact the patient or family anonymously through the company with the database and search engine in order to have discussions about the patient's treatment, outcomes, etc. The patient or family member receiving the communication from the treating institution would be told that they were under no obligation to identify him(her)self to the inquiring client, and in fact did not have to reply to the letter if they did not wish to go any further. If the patient or family member agree to communicate, then they could reply via e-mail back to the sending institution, who would forward the e-mail to the proprietors of the database who would forward the communication back to the inquiring client. This would all be performed anonymously to protect the privacy of both the client and the patient or family.

The scope of the proposed automatic clustering system need not be restricted to groups of those patients who submitted their own medical information. The users of the system may also include other individuals and institutions associated with the health care industry, such as radiologists, physicians, clinical researchers, and pharmaceutical companies. In addition to the patient-patient interactions facilitated by the clustering system, other combinations of interactions between individuals are made possible, such as patient-physician, patient-researcher, physician-radiologist, etc. For example, a clinical researcher investigating multiple sclerosis (MS) is likely to submit many medical images of patients afflicted with the condition. When an MS patient submits his or her own scan, and it matches other scans containing MS lesions, the patient and clinical researcher will be grouped together, opening a new communication channel.

Referring now to FIG. 15, the disclosed system and process 1550 can also be used in a clinical trial setting, where patients searching for alternative treatment options for their disease or condition may consider turning to clinical trials of experimental drugs or treatments. The medical image database coupled with the self-organizing groups provides a means of connecting these patients with institutions performing clinical trials. The patient or hospital connect to the server, step 1551, and submits a patient's medical information, e.g., a scan and additional data, to the database, step 1552, where the matching medical images are returned with possible diagnoses 1553. Based on the matching diagnoses, it is checked if an appropriate clinical trial is currently registered with the database, step 1554. If the answer is affirmative, the patient or physician has the option of initiating a communication path with the investigators of the clinical trials. Upon consent, the clinical researchers would have anonymous access to the medical images of the patient interested in clinical trials, step 1555, in order to evaluate whether the patient matches the requirements for the trial. By matching the patient's medical images to the database, clinical researchers interested in a certain condition and patients afflicted with that condition seeking care alternatives are automatically clustered into the same group, step 1556. This approach can significantly broaden the number of candidates that can be enlisted in a clinical trial.

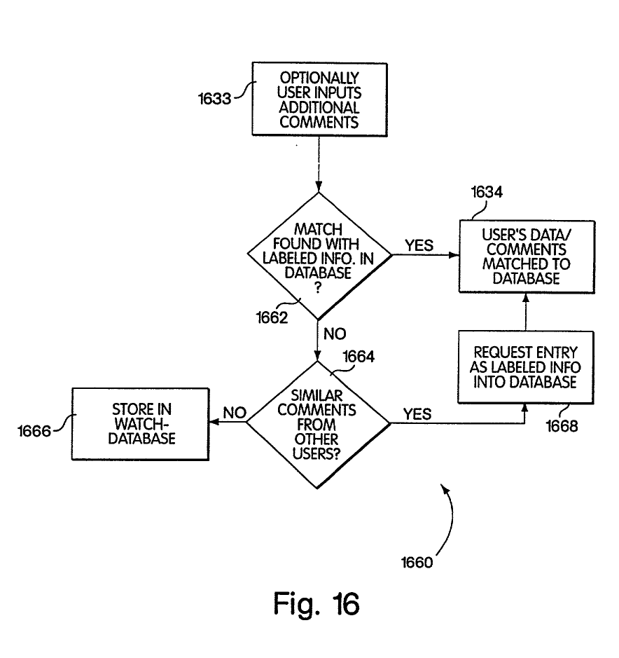

The medical information submitted by the users who were grouped together include uninterpreted as well as interpreted information. Interpreted information refers to information in the database that has been “labeled” or associated with a diagnosis. Conversely, uninterpreted information include patient information that has not been associated with a diagnosis, but may also include information supplied by the patient as a comment and for which no entry exists yet in the database. Referring back to FIG. 12, uninterpreted information is represented by the areas of 1201 and 1202 that do not overlap with any of the exemplary diagnoses D₁, D₂ and D₃. However, such uninterpreted information can be useful for forming groups and associating users with existing groups. For example, the point (X) in FIG. 12 is shared by multiple users and can be used to form a group that includes two users, even if a diagnosis has not yet been established. Such uninterpreted information that can originate from user-supplied comments, can be stored in a “Watch Database” at the server, as indicated as step 1666 in process 1660 depicted in FIG. 16.

As described above with reference to FIG. 13, a user/patient may input in step 1633 additional comments, which can be text, measurement data and/or image information, that could be provided as an answer to a question: “Do you have any other symptoms?” Referring now back to FIG. 16, the system will check if language or features in the comment can be associated with “labeled” information in the database, step 1662. If this is the case, then the user's comments are matched up with database entries, step 1634. If the database has no corresponding entries, then the databases, including the “Watch Database”, are searched for similar comments from other users, step 1664. If no similar comments are found, the user's comment is entered into the “Watch Database” for future comparisons, step 1666. If corresponding entries are found in step 1664, then it appears likely that the hitherto unlabeled feature is important in context of a medical diagnosis, and the process 1660 may request or at least suggest that this unlabeled feature be included and “labeled” in the database, step 1666. Techniques for arriving at criteria for two features (text, images, etc.) to be viewed as being similar are known in the art.

In summary, grouping invokes a bootstrapping technique where the initial collection of information has been labeled or interpreted, whereas the submitted queries are usually “unlabeled”. Once two unlabeled queries are matched to similar labeled information in the database, these unlabeled queries are interpreted, and a group is formed based on both the labeled and the originally unlabeled information. The automatic grouping approach provides a multitude of advantages to existing subjective methods of forming interest groups that rely heavily on the individuals being able to precisely and accurately express exactly where their interests lie.

Thus, while the invention has been disclosed in connection with the preferred embodiments shown and described in detail, various modifications and improvements thereon will become readily apparent to those skilled in the art. It should be understood that all matter contained in the above description or shown in the accompanying drawings shall be interpreted as illustrative, and not in a limiting sense, and that the following claims should be interpreted in the broadest sense allowable by law.

What is claimed is:

1. A method comprising:
receiving a plurality of images, each one of the plurality of images including an instance of a human body part obtained through a medical imaging technique;
registering each one of the plurality of images in a non-rigid manner to a coordinate system to superimpose one or more like features within each one of the plurality of images within the coordinate system, thereby obtaining a plurality of registered images;
labeling each one of the plurality of registered images with a label according to an observed characteristic;
obtaining one or more feature vectors from each one of the plurality of registered images; and
training a model to associate the one or more feature vectors with the label.

2. The method of claim 1 further comprising applying the model to generate a label for each one of an additional plurality of images.

3. The method of claim 1 wherein each label is a position of an image on a z-axis perpendicular to an image plane of the image.

4. The method of claim 1 wherein each label is a region of interest.

5. The method of claim 1 wherein each label is at least one of an age, a sex, a diagnosis, a presence of contrast agents, an image type, or a diagnostic significance of a region of interest.

6. The method of claim 1 wherein the model is derived from a statistical learning methodology.

7. The method of claim 1 wherein the model is a linear regression model.

8. The method of claim 1 wherein partial least squares are used to determine one or more coefficients of the model.

9. The method of claim 1 wherein the model includes a weighted norm that is a function of a type of the label, the weighted norm used to estimate new labels with the model, and the type of label including at least one of a pathology, a spatial position, or client data.

10. The method of claim 1 further comprising applying the model to generate labels for a second database of images.

11. The method of claim 1 wherein each one of the plurality of images includes at least one of a magnetic resonance image or a computerized tomography image.

12. The method of claim 1 wherein each one of the plurality of images includes at least one of a head image, a neck image, a spine image, a chest image, or a musculo-skeletal image.

13. The method of claim 1 further comprising:
locating an image database that is accessible through a network, the image database including a second plurality of images;
registering each one of the second plurality of images in a non-rigid manner to a coordinate system to superimpose one or more like features within each one of the second plurality of images within the coordinate system, thereby obtaining a second plurality of registered images;
applying the model to label each one of the second plurality of images; and
searching the image database using the labels to evaluate a similarity of a query to one or more of the second plurality of images.

14. The method of claim 11 further comprising organizing a plurality of databases by locating images, registering images, and labeling images within each one of the plurality of databases, each one of the plurality of databases being labeled with a different model, and searching the plurality of databases using the labels to evaluate a similarity of a query to one or more records in each of the plurality of databases.

15. A system comprising:
receiving means for receiving a plurality of images, each one of the plurality of images including an instance of a human body part obtained through a medical imaging technique;
registering means for registering each one of the plurality of images in a non-rigid manner to a coordinate system to superimpose one or more like features within each one of the plurality of images within the coordinate system, thereby obtaining a plurality of registered images;
labeling means for labeling each one of the plurality of registered images with a label according to an observed characteristic;
obtaining means for obtaining one or more feature vectors from each one of the plurality of registered images; and
training means for training a model to associate the one or more feature vectors with the label.

16. A computer program product comprising:
computer executable code for receiving a plurality of images, each one of the plurality of images including an instance of a human body part obtained through a medical imaging technique;
computer executable code for registering each one of the plurality of images in a non-rigid manner to a coordinate system to superimpose one or more like features within each one of the plurality of images within the coordinate system, thereby obtaining a plurality of registered images;
computer executable code for labeling each one of the plurality of registered images with a label according to an observed characteristic;
computer executable code for obtaining one or more feature vectors from each one of the plurality of registered images; and
computer executable code for training a model to associate the one or more feature vectors with the label.

17. The computer program product of claim 16 further comprising computer executable code for applying the model to generate a label for each one of an additional plurality of images.

18. A method comprising:
receiving a plurality of images, each one of the plurality of images including an instance of a human body part obtained through a medical imaging technique;
registering each one of the plurality of images in a non-rigid manner to a coordinate system to superimpose one or more like features within each one of the plurality of images within the coordinate system, thereby obtaining a plurality of registered images;
receiving a header for each one of the plurality of images that includes data associated with the one of the plurality of images;
obtaining one or more feature vectors from each one of the plurality of registered images;
training a model to associate the one or more feature vectors with the header; and
applying the model to identify the presence of any errors in a new header for a new image.

19. The method of claim 16 wherein the header includes a magnetic resonance imaging characteristic.

20. The method of claim 18 wherein the header includes at least one of a contrast agent attribute that indicates a presence or absence of a contrast agent, or a sequence type attribute that indicates a sequence type for the plurality of images, the sequence type being at least one of MRA or T1.

21. A method comprising:
receiving a plurality of images, each one of the plurality of images including an instance of a human body part obtained through a medical imaging technique;
registering each one of the plurality of images in a non-rigid manner to a coordinate system to superimpose one or more like features within each one of the plurality of images within the coordinate system, thereby obtaining a plurality of registered images;
obtaining one or more feature vectors from each one of the plurality of registered images;
associating a pathology with each one of the plurality of images;
training a model to associate the pathology associated with each image with the one or more feature vectors for that image; and
applying the model to identify the presence of the pathology in a new image.

22. The method of claim 21 further comprising obtaining each one of the one or more feature vectors from a region of interest within one of the plurality of images.

23. A method comprising:
receiving a plurality of images, each one of the plurality of images including an instance of a human body part obtained through a medical imaging technique;
registering each one of the plurality of images in a non-rigid manner to a coordinate system to superimpose one or more like features within each one of the plurality of images within the coordinate system, thereby obtaining a plurality of registered images;
identifying one or more regions of interest in each of the plurality of registered images, the regions of interest including a pathology; and
generating a spatial probability map of locations of the pathology from the plurality of registered images and the regions of interest.

24. The method of claim 1 further comprising using the spatial probability map as a medical diagnostic aid.
